SEQUENCE LISTING (10 sequences)

SEQ ID NO: 1 - protein, 114 aa, Canis
  UNSURE 95..109
EVQLVESGGDLVKPGGSLRLSCVASGFTFSSYHMSWVRQAPGKGLQWVAYINSGGSSTSYADAVKGRFTISRDNAKNTLYLQMNSLRAEDTAVYXXXXXXXXXXXXXXXGQGTL

SEQ ID NO: 2 - protein, 114 aa, Canis
QVQLVESGGDLVRPGGSLRLSCVASGFTFSYYHMNWVRQAPGKGLQWVAYINSGGEITTFVDAVKGRFTISRDNAKNTLYLQMNSLRAEDTAIYYCAREDSSTWGFDYWGQGIL

SEQ ID NO: 3 - protein, 114 aa, Canis
EVQLVESGGDLVKPGGSLRLSCVASGFTFSSYHMSWVRQAPGKGLQWVAYINSGGSSTSYADAVKGRFTISRDNAKNTLYLQMNSLRAEDTAVYYCAREDSSTWGFDYWGQGTL

SEQ ID NO: 4 - protein, 114 aa, Canis; identical to SEQ ID NO: 2

SEQ ID NO: 5 - protein, 114 aa, Canis
EVQLVESGGDLVKPGGSLRLSCVASGFTFSSYHMSWVRQAPGKGLQWVAYINSGGSSTSYADAVKGRFTISRDNAKNTLYLQMNSLRAQDTAVYYCAREDSSTWGFDYWGQGTL

SEQ ID NO: 6 - protein, 108 aa, Canis
  UNSURE 86..99
SYVLTQLPSKNVTLKQPAHITCGGDNIGSKSVHWYQQKLGQAPVLIIYYDSSRPTGIPERFSGANSGNTATLTISGALAEDEADYXXXXXXXXXXXXXXGEGTQLTVL

SEQ ID NO: 7 - protein, 108 aa, Canis
SYVLTQLPSKNVTLKQPAHITCGGDNIGSKSVHWYQQKLGQAPVLIINYDTARPTGIPERFSGANSGNTATLTISGALAEDEADYYCQVWDSSAKAWVFGEGTQLTVL

SEQ ID NO: 8 - protein, 108 aa, Canis; identical to SEQ ID NO: 7

SEQ ID NO: 9 - protein, 108 aa, Canis
SYVLTQLPSKNVTLKQPAHITCGGDNIGSKSVHWYQQKLGQAPVLIIYYDSSAPTGIPERFSGANSGNTATLTISGALAEDEADYYCQVWDSSAKAWVFGEGTQLTVL

SEQ ID NO: 10 - protein, 108 aa, Canis; identical to SEQ ID NO: 7

(19) United States
(12) Patent Application Publication
(10) Pub. No.: US 2024/0200127 A1
(43) Pub. Date: Jun. 20, 2024

(54) A METHOD FOR IDENTIFYING LEAD SEQUENCES

(71) Applicant: PETMEDIX LTD., Cambridge (GB)

(72) Inventors: Albert Vilella BERTRAN, Cambridge (GB); Daniel John BOLLAND, Cambridge (GB)

(21) Appl. No.: 18/285,822

(22) PCT Filed: Apr. 6, 2022
(86) PCT No.: PCT/EP2022/059150

§ 371 Date: Oct. 5, 2023

(30) Foreign Application Priority Data
Apr. 7, 2021 (GB) 2104943.2

(51) Int. Cl. C12Q 1/6869

(52) U.S. Cl. CPC C12Q 1/6869

(57) ABSTRACT

A method for identifying lead sequences for antibody or T cell receptor expression, the method comprising providing a single sample B or T cells derived from a host, performing a single sequencing step to sequence nucleic acid from the single sample, and selecting a lead sequence for expression.

The present invention relates to a method for identifying lead sequences, for example for antibody or T cell receptor expression.

### BACKGROUND

When an antigen encounters the immune system of a host, it reacts with and activates a complementary B-lymphocyte (B cell). This B-lymphocyte then rapidly proliferates to produce a large number of clones in a process referred to as clonal expansion. During this process, the B-lymphocyte undergoes affinity maturation as a result of somatic hypermutations. The B-lymphocytes clones each produce unique antibodies that bind to the invading antigen, targeting it for destruction. T cells similarly undergo clonal expansion and affinity maturation of the T cell receptor.

The identification of antibody lead sequences is part of the discovery process for e.g. new antibody-based and TCR-based therapeutics. It involves the interrogation of early stage ‘hit’ molecules to establish whether said molecules are structurally and functionally suitable for the next stage of drug discovery.

In the art, methods for selecting lead antibody sequences include analysing B cells isolated from a host that has been immunised with a target antigen of interest. The analysis generally takes sequences from single cell B cells only, since these cells are guaranteed to contain paired heavy and light chain sequences for expression of the antibody. The same is true of T cells.

There is still a need for improved methods for identification and selection of lead antibody and TCR sequences.

### SUMMARY OF THE INVENTION

Provided herein is a method for identifying a lead antibody sequence, the method comprising:
i. providing a single sample of B cells derived from a spleen and/or bone marrow tissue, wherein the sample comprises intact and fragmented B cells;
ii. performing a single sequencing step to sequence nucleic acid from the single sample, to identify paired antibody heavy and light chain sequences from intact cells and nucleic acid sequences encoding antibody chains that are not from intact cells, and
   selecting a heavy or light chain lead sequence for antibody expression, wherein the heavy or light chain lead sequence forms part of a cluster of homologous sequences, and the cluster comprises at least one heavy or light chain sequence which is not from an intact cell.

Also provided herein is a method for identifying a lead T cell receptor (TCR) sequence, the method comprising:
i. providing a single sample of T cells derived from a thymus and/or bone marrow tissue, wherein the sample comprises intact and fragmented T cells;
ii. performing a single sequencing step to sequence nucleic acid from the single sample, to identify paired TCR heavy and light chain sequences from intact cells and nucleic acid sequences encoding TCR chains that are not from intact cells, and
   selecting a heavy or light chain lead sequence for TCR expression, wherein the heavy or light chain lead sequence forms part of a cluster of homologous sequences, and the cluster comprises at least one heavy or light chain sequence which is not from an intact cell.

In some embodiments, for the B cells, the spleen and/or bone marrow tissue is derived from a rodent that has been immunised with a target antigen, and for the T cells, the thymus and/or bone marrow tissue is derived from a rodent that has been immunised with a target antigen.

In some embodiments, in step i), the B or T cells are sorted, optionally counted, and spun down for pelleting.

In some embodiments, the cells are sorted via FACS or MACS.

In some embodiments, in step i), the intact and fragmented B or T cells are encapsulated into emulsion particles, optionally into microfluidic drops, wherein the sample comprises a mixture of encapsulated intact cells and encapsulated nucleic acid from the fragmented B or T cells, optionally wherein the encapsulated nucleic acid from the fragmented B cells encodes an antibody heavy or light chain and the encapsulated nucleic acid from the fragmented T cells encodes a TCR heavy or light chain.

In some embodiments, the nucleic acid in the sample is RNA.

In some embodiments, in step ii), the nucleic acid is sequenced via a next-generation sequencing instrument.

In some embodiments, after step ii) an antibody or TCR chain that is not from an intact cell is partnered with a heavy or light chain from a paired heavy and light chain from an intact cell.

In some embodiments, the method further comprises comparing the amino acid sequences of the antibody or TCR chains that are not from intact cells with the paired antibody or TCR heavy and light chain sequences from intact cells, and selecting a heavy or light chain from a paired sequence to partner with a nucleic acid sequence encoding an unpaired antibody or TCR chain that is not from an intact cell, wherein the corresponding heavy or light chain from said paired sequence is at least 90% homologous to the amino acid sequence of the unpaired antibody or TCR chain.

In some embodiments, sequences that are derived from the same precursor B or T cell are clustered together in a single cluster, and/or wherein sequences with an amino acid sequence homology of 90% or more across the variable heavy or variable light domains are clustered.

In some embodiments, the cluster comprises at least one heavy and one light chain that are not from intact cells, optionally wherein the cluster further comprises at least one heavy and light chain from intact cells, or wherein the cluster further comprises at least one heavy and light chain from intact cells.

In some embodiments, the heavy or light chain lead sequence is selected from a heavy or light chain sequence that is not from an intact cell, or wherein the heavy or light chain lead sequence is selected from a heavy or light chain sequence is from an intact cell.

In some embodiments, the method further comprises expressing the heavy and light chains lead sequences together in a cell to generate an antibody or TCR, optionally further formulating with a pharmaceutically acceptable excipient or carrier to from a pharmaceutical composition.

In some embodiments, in step i) the cells in the sample are bound to an oligo-tagged antibody or fragment thereof.

In some embodiments, the cell sample is from tissue from one or more hosts, wherein the tissue from each host is associated with a different oligo-tagged antibody.

In some embodiments, step ii) further comprises determining the level of oligo associated with each cell in the sample, to identify paired antibody heavy and light chain sequences from intact cells and nucleic acid sequences encoding antibody chains that are not from intact cells.

In some embodiments, step ii) further comprises determining the level of oligo association and V(D)J expression for each cell in the sample, optionally wherein the levels of oligo association and V(D)J expression of each cell in the sample is assessed to determine the relative levels of oligo association and V(D)J expression, to identify paired antibody heavy and light chain sequences from intact cells and nucleic acid sequences encoding antibody chains that are not from intact cells.

### BRIEF DESCRIPTION OF THE FIGURES

FIG. 1 shows a schematic of an exemplary method of the invention.

FIG. 2 shows a schematic of an encapsulation process in a method of the invention. B cell are initially intact, become fragmented upon processing, and are subsequently encapsulated (GPON encapsulation method (GEM). Each encapsulate may be empty, or contain a B cell or B cell fragment.

FIG. 3 shows a schematic of a clustering process used in a method of the invention. Each circle represents an intact or reconstructed cell with a paired heavy and light chain. Clusters are as depicted by the grouping of single cells.

FIG. 4 shows the output from a sequencing step in a method of the invention, where the sequences of four heavy chain amino acid sequences and four light chain amino acid sequences are determined. These sequences may be compared to a germline reference, to identify mutations in each chain. These sequences may also be used to determine complimentary pairings for unpaired heavy or light chains in the sample.

FIG. 5 shows a schematic of an exemplary method for hashtagging cells in a method of the invention.

FIG. 6 and FIG. 7 show the results from V(D)J expression analyses (top graphs) and oligo expression analyses (bottom graphs) in a method of the invention. The UMI count measures the number of observed transcripts (either V(D)J or oligo) in the sample. The barcode is a tag for each individual cell in the sample. A down-step in the graph trend (in some cases indicated by an arrow) indicates a likely change in cell population. Barcodes (cells) with a high UMI count indicates an intact cell, and barcodes with a low UMI count indicates a fragmented cell. The results of each graph may be compared and correlated (see dotted lines between the graphs) to more accurately determine intact and fragmented cells. In the bottom graph of FIG. 7, the area under the curve may be correlated with the down-step of both the oligo expression and V(D)J graphs to determine whether a sample comprises non-B cells. The second shaded quadrant (indicated by an arrow) indicates barcodes in the sample that may not be B cells.

FIG. 8 shows a schematic of an exemplary method of the invention, including a hashtagging process. ‘Diva’ cells are those that are more difficult to characterise as either intact or fragmented cells. Diva cells may be plasma cells, for example, which are fragile and thus more prone to fragmentation. The hashtagging method allows for these cells to be more accurately characterised.

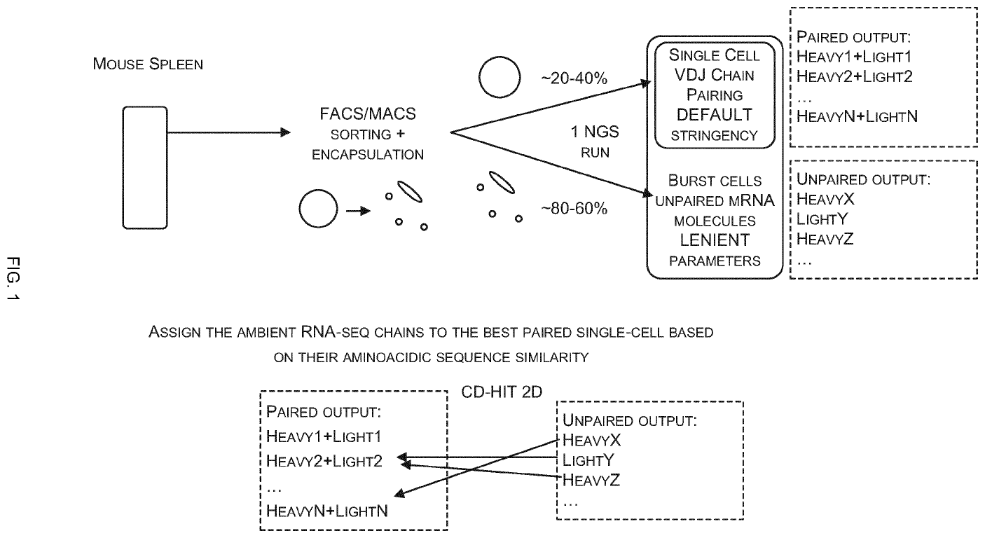

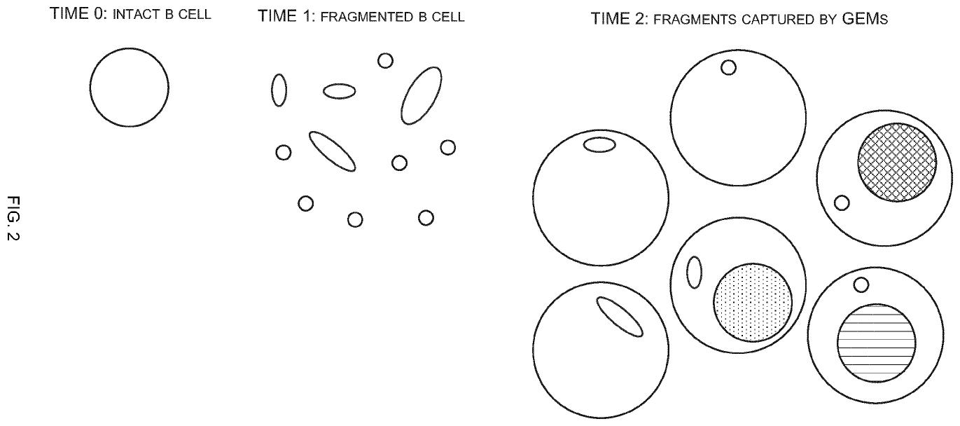

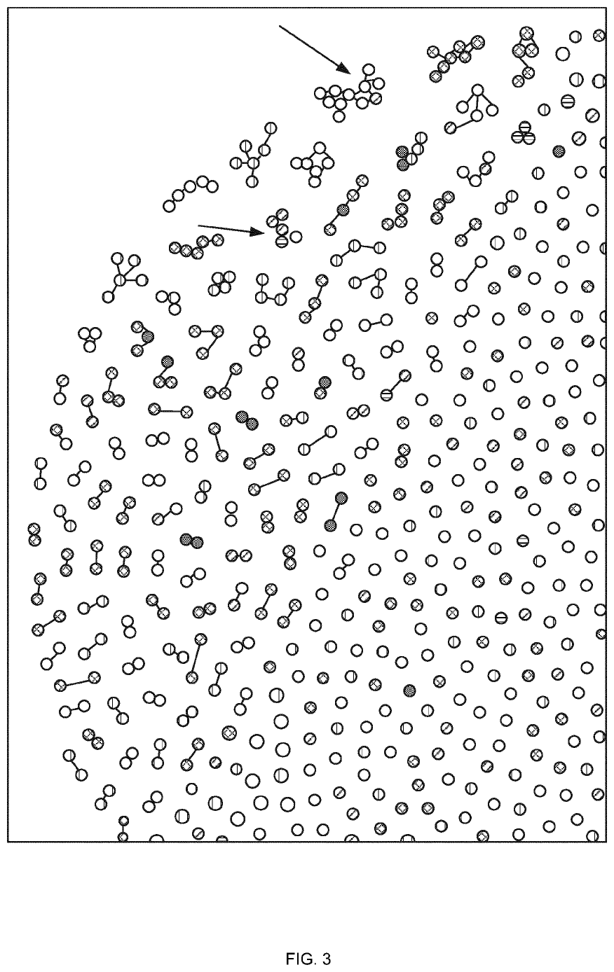

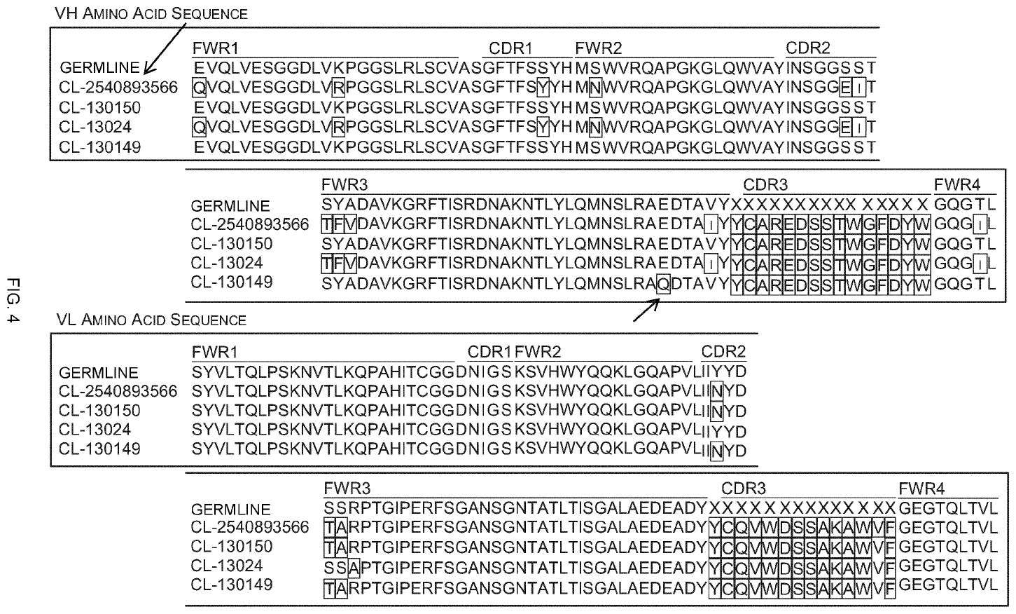

### DETAILED DESCRIPTION OF THE INVENTION

The present invention provides an improved method, such as a high-throughput method, for identifying lead antibody sequences, by analysing both whole and lysed B cells from a tissue sample.

Similarly, the whole approach can be applied to T cells for identification of T cell receptor leads. It will be appreciated that the mechanism by which B-cell antigen receptors are generated is similar to that of T cells, and so any teaching in the methods herein in respect of antibodies (having a heavy and light chain) applies analogously to the TCR which also contains V and J gene segments in the TCR alpha locus, and V, D and J gene segments for the TCRB locus. Certain aspects of the invention may be described only in respect of antibodies, but apply equally to the TCR selection.

Therefore, the present invention provides an improved method, such as a high-throughput method for identifying lead TCR sequences, by analysing both whole and lysed T cells from a host tissue sample.

When single cells are processed, some break up or lyse, with the nucleic acid contents of these cells (e.g. heavy and light chain sequences) becoming unlinked from the original cell. There is therefore a potential loss of information regarding the antibodies and TCRs found in the full set of antigen-specific cells from the host. Sequences of some somatic hypermutated B and T cell clones may have only been present in cells that lysed upon analysis.

Commonly, this sequence information from lysed cells is not considered. In the present invention the additional sequence information in the nucleic acid found not in an intact cell is used in the selection of antibody and TCR leads.

Further, the methods described herein enable a heterogenous mixture of intact and lysed cells to be analysed from a single sample and in a single sequencing step, rather than conducting multiple parallel sequencing steps, which can be inefficient, costly and increase the likelihood of errors.

The present invention thus relates to a method for identifying a lead antibody sequence, the method comprising:
i. providing a single sample of B cells derived from a spleen and/or bone marrow tissue, wherein the sample comprises intact and fragmented B cells;
ii. performing a single sequencing step to sequence nucleic acid from the single sample, to identify paired antibody heavy and light chain sequences from intact cells and nucleic acid sequences encoding antibody chains that are not from intact cells, and
   selecting a heavy or light chain lead sequence for antibody expression, wherein the heavy or light chain lead sequence forms part of a cluster of homologous sequences, and the cluster comprises at least one heavy or light chain sequence which is not from an intact cell.

The present invention also relates to a method for identifying a lead T cell receptor (TCR) sequence, the method comprising:
i. providing a single sample of T cells derived from a thymus and/or bone marrow tissue, wherein the sample comprises intact and fragmented T cells;
ii. performing a single sequencing step to sequence nucleic acid from the single sample, to identify paired TCR heavy and light chain sequences from intact cells and nucleic acid sequences encoding TCR chains that are not from intact cells, and
   selecting a heavy or light chain lead sequence for TCR expression, wherein the heavy or light chain lead sequence forms part of a cluster of homologous sequences, and the cluster comprises at least one heavy or light chain sequence which is not from an intact cell.

In one aspect, the method of the invention may be carried out using any antibody-producing tissue. In an embodiment, the tissue may be spleen tissue and/or bone marrow tissue.

In one aspect, the method of the invention may be carried out using any T cell producing tissue. In an embodiment, the tissue may be thymus gland tissue and/or bone marrow.

Further, the method of the invention may be carried out using a tissue sample from a human or animal. In an embodiment, the tissue sample may be from a host, where the host may be a human or animal, for example a rodent (such as a mouse), dog, cat or horse.

In one embodiment, the host is a human.

In one embodiment, the host is a dog.

In one embodiment, the host is a cat.

In one embodiment the method comprises immunising the host (e.g. a mouse) with a target antigen of interest. This activates certain B cells and initiates the process of B cell clonal expansion. Clonal expansion produces a variety of mutant B cell clones via somatic hypermutation, which creates random mutations in the V(D)J variable region genes comprised by the B cell. Likewise, T cell clonal expansion is activated by immunisation.

### B or T Cell Isolation, Sorting and Encapsulation

The method of the invention comprises providing a sample of B cells or T cells.

After immunisation with an antigen of interest, the spleen and/or bone marrow of the immunised host (e.g. a mouse) may be extracted. Cells from the extracted tissue may be homogenized and sorted to select B cells and/or plasmablasts (plasma cells). Alternatively or in addition, after immunisation with an antigen of interest, the thymus gland tissue and/or bone marrow of the immunised host (e.g. a mouse) may be extracted. Cells from the extracted tissue may be homogenized and sorted to select T cells.

In some embodiments, the method comprises sorting the extracted cells via a cell sorting technique such as Fluorescence Cell Sorting (such as FACS) or Magnetic Cell Sorting (such as MACS). These processes exclude dead cells and ensure a homogenous sample of the biological B or T cells.

In some embodiments, the method comprises counting the number of cells after sorting, such as via a cell counting instrument. This provides an estimate of the number of B cells or T cells present in the sample.

The sorted cells may then be spun down for pelleting and encapsulated. Thus, in some embodiments, the B or T cells are sorted, optionally counted, and spun down for pelleting.

In some embodiments, the sorted cells are encapsulated into emulsion particles, optionally into microfluidic drops.

Encapsulation into microfluidic drops enables formation of an emulsion particle encapsulating a single particle/cell with a labelled gel bead along with reverse transcriptase (RT) reagents. The RT reagents include primers for, e.g. human, mouse or dog, heavy and light chains. These primers allow amplification and labelling, e.g. with barcodes, to enable each cell's transcriptome (e.g. heavy and light chain mRNAs) to be indexed. In this way, thousands of cells per sample may be barcoded and prepped for analysis. A number of suitable methods for encapsulating a single particle/cell with labelled reagents are known and include, for example, a gravity-based approach such as Celsingle™ technology (Celsee), Fluidigm C1 or Polaris System, 10× Genomics Chromium Single Cell Immune Profiling instrument, Takara iCell8 system, 1CellBio inDrop system, Dolomite Bio Nadia system, Becton Dickinson Rhapsody system, MissionBio Tapestri system, Bio-Rad ddSEQ or Celsee Genesis system, Cell Microsystems CellRaft AIR system, Vycap Puncher Platform system, ALS AVISO CellCelector system or Menarini Silicon Bio DEEPArray N×T system. Suitable methods for immune profiling may be provided using specific reagents in accordance with the manufacturer's protocol e.g using a 5′VDJ kit.

Processing the cells by sorting and encapsulation causes a fraction of the cells to burst. This is a particular problem for fragile cells that are more prone to breaking, such as plasma cells. Thus, the single cell sample comprises both intact B or T cells and fragmented B or T cells, i.e. ‘free’ nucleic acid. The cell sample is not split to separate intact cells from fragmented cells. This ‘free’ nucleic acid may be called ambient mRNA herein, or may be also referred to as nucleic acid not from intact cells. For a B cell sample, the intact cells comprise paired heavy and light chain antibody sequences, and for a T cell sample, the intact cells comprise paired heavy and light chain TCR sequences. This pairing is lost in the fragmented cells. The free nucleic acid comprises unpaired heavy and light chain sequences, with no marker for determining which original B or T cell clone said sequences were derived from.

Thus, in some embodiments, the sample comprises a mixture of encapsulated intact B or T cells and encapsulated nucleic acid (or ambient mRNA) from fragmented B or T cells, wherein the encapsulated intact B or T cells comprise a paired heavy and light chain sequence, and the encapsulated nucleic acid from fragmented B or T cells encodes an antibody or TCR heavy or light chain. Each single-cell sequence pair corresponds to an individual B or T cell clone. In some embodiments, the cells are plasma cells.

In some embodiments, the method further comprises estimating the ratio of intact to fragmented cells that undergo encapsulation. If the number of cells reported to be sorted by the FACS/MACS process is a number of events ‘N’, it is possible to take an aliquot of these and estimate the number of intact cells via a cell counting instrument, which gives the number ‘R’ of remaining intact cells after counting, where ‘R’ will always be smaller or equal to ‘N’. It is possible to use the ‘R’ to ‘N’ ratio as an estimate of the ratio of intact to fragmented cells that undergo encapsulation.

### Sequencing and Pairing

The method of the invention comprises sequencing nucleic acid from the sample, to identify paired heavy and light chain sequences from intact cells, and nucleic acid sequences encoding antibody or TCR chains that are not from intact cells.

Whilst the sample is not split to separate intact cells from fragment cells, it will be appreciated that the sample may be separated into aliquots for sequencing, due to e.g. any sample volume constraints, but each aliquot will contain intact and fragmented B or T cells, and each aliquot will undergo a single sequencing step.

In some embodiments, the nucleic acid for sequencing is RNA, particularly mRNA.

In some embodiments, the method comprises preparing a next-generation sequencing DNA library from the mixed set of heavy and light chains comprised by the encapsulates, optionally from the barcoded encapsulates. The libraries may then be then sequenced through a next-generation sequencing instrument, which supports wide-scale parallel sequencing.

In some embodiments, after sequencing the resulting encapsulates, an estimate of the total amount of encapsulates is be recorded and compared to the number of counted cells before encapsulation.

In some embodiments the sequence outputs are analysed to determine whether the encapsulates comprise a heavy and light chain pair, or just a heavy chain, or just a light chain, optionally based on the V(D)J expression level of each cell in the sample, for example via a set of analysis pipelines that process single-cell RNA-seq output to align or assemble reads into full length sequences, generate feature-barcode matrices and perform clustering and gene expression analysis, such as provided in Cell Ranger software (10× Genomics), for example. Methods of determining V(D)J expression are well-known in the art, and any suitable method may be used.

The V(D)J expression level of an encapsulate is an indicator of the presence of heavy and light chains, where a high expression level suggests the presence of a paired heavy and light chain in the cell, and a low expression level suggests the presence of a single heavy or light chain in the encapsulate.

In some embodiments, the level of VDJ expression for each cell in the sample is determined, to identify paired antibody heavy and light chain sequences from intact cells and nucleic acid sequences encoding antibody chains that are not from intact cells.

In some embodiments, the level of VDJ expression for each cell in the sample is determined, wherein the expression levels are compared to determine the relative expression levels (i.e. high and low) of each cell in the sample.

Software such as Cell Ranger (10× Genomics) analyses FASTQ files, or similar text-based format for storing both a biological sequence (usually nucleotide sequence) and its corresponding quality scores, which are then are aligned or assembled and further analysed to generate single-cell V(D)J sequences and annotations for a single library. Thus, in some embodiments the sequence outputs are converted into FASTQ files, and the level of VDJ expression measured to identify paired heavy and light chain sequences, heavy chain sequences, and light chain sequences.

In some embodiments, the results of these analyses are inputted into a computational tool for clonal grouping, such as enclone software (10× Genomics), using both stringent parameters and lenient parameters. The stringent parameters classify heavy and light chain sequence pairs for validated intact cells with a corresponding barcode. The lenient parameters classify all material, including one chain clonotypes (heavy or light) with a corresponding barcode for the non-intact cells. In some embodiments, heavy and light chain sequence pairs are then classified and translated into amino acid sequences.

So that the sequence information from the unpaired heavy and light antibody chains is not lost, said sequences may be paired with a corresponding heavy or light chain sequence, that will ultimately result in the expression of a functioning antibody or TCR. This process effectively reconstructs the original burst B cell clone. Thus, in some embodiments, the method comprises reconstructing from non-intact B or T cell clones by pairing unpaired heavy or light antibody chains with a corresponding heavy or light chain sequence that results in the expression of a functioning antibody or TCR.

In one embodiment, an antibody or TCR chain that is not from an intact cell is partnered with a heavy or light chain from a paired heavy and light chain from an intact cell.

In order to pair the unpaired chains with the correct corresponding chain that will result in the expression of a functioning antibody or TCR, the sequence of an unpaired chain is compared with those of the paired chains. An unpaired chain is identified as a heavy or light chain and, depending on amino acid sequence homology to a heavy or light chain of a paired sequence, paired with the corresponding chain of that pair, thereby forming a reconstructed heavy and light chain pair.

Thus, in some embodiments, the method comprises comparing the amino acid sequences of the antibody chains that are not from intact cells with the paired antibody heavy and light chain amino acid sequences from intact cells, and selecting a heavy or light chain from a paired sequence to pair with an amino acid sequence of an unpaired antibody chain that is not from an intact cell, wherein the corresponding heavy or light chain from said paired sequence is at least 90% homologous to the amino acid sequence encoding the unpaired antibody chain, such as at least 91%, 92%, 93%, 94%, 95%, 96%, 97%, 98% or 99% homologous. In some embodiments, the corresponding heavy or light chain from said paired sequence is less than 100% homologous to the amino acid sequence encoding the unpaired antibody chain.

In one embodiment, the method comprises comparing an unpaired heavy chain sequence with a paired heavy and light chain sequence, and if a sequence homology of 90% or more is identified with the paired heavy chain, such as at least 91%, 92%, 93%, 94%, 95%, 96%, 97%, 98% or 99% homology, pairing the unpaired heavy chain with the light chain from said pair. In another embodiment, the method comprises comparing an unpaired light chain sequence with a paired heavy and light chain sequence, and if a sequence homology of 90% or more is identified with the paired light chain, such as at least 91%, 92%, 93%, 94%, 95%, 96%, 97%, 98% or 99% homology, pairing the unpaired light chain with the heavy chain from said pair. In one embodiment, the method comprises conducting both of these comparisons, optionally for all unpaired chains in the sample. Here, sequence homology relates to amino acid sequence homology. In some embodiments, the sequence homology is less than 100% homology.

In some embodiments, this pairing is carried out using a software program for clustering and comparing protein or nucleotide sequences such as CD-HIT 2D software (Weizhong Li's Group, Sanford-Burnham Medical Research Institute). This software compares two datasets (i.e. the paired and unpaired sequences), and identifies the sequences in dataset 2 that are similar to dataset 1 above a specified threshold, such as 90% protein sequence identity.

i. Clustering Paired Sequences

The method of the invention may optionally further comprise clustering or grouping paired sequences. Clustering the sequences allows for the most successful B or T cell lineages to be identified.

A B and T cell clonal family is generally defined by the use of related immunoglobulin heavy chain and/or light chain V(D)J sequences. Clones with different V(D)J segment usage usually exhibit different binding characteristics. Thus, related immunoglobulin heavy chain V(D)J sequences can be identified by their shared usage of V(D)J gene segments encoded in the genome. For example, antibody sequences expressed by individual B cells may be arranged by heavy-chain V-gene family usage and clustered to generate phylogenetic trees.

Thus, in some embodiments, the method comprises clustering related amino acid sequences based on at least one characteristic of the sequences.

In some embodiments, the method comprises clustering amino acid sequences derived from the same phylogenetic lineage i.e. derived from the same precursor B or T cell. In some embodiments, the method comprises clustering heavy chain sequences that are derived from the same precursor B or T cell. In some embodiments, the method comprises clustering light chain sequences that are derived from the same precursor B or T cell. In some embodiments, the method comprises clustering paired heavy and light chain sequences from either intact or reconstructed cells, wherein the heavy and light chains are derived from the same precursor B or T cell.

This generates a series of clonal family clusters.

Clusters may comprise paired heavy and light chain sequences from intact cells, or from a mixture of intact cells and not from intact cells (i.e. the reconstructed cells). In the method of the invention, the cluster comprises at least one paired heavy and light chain sequence not from an intact cell.

Within a clonal family, there are generally subfamilies that vary based on shared mutations within their V(D)J segments, that can arise during B or T-cell gene recombination and somatic hypermutation. Clones with the same V(D)J segment usage but different mutations exhibit different binding characteristics. B and T cells undergo somatic hypermutation, where random changes in the nucleotide sequences of the antibody genes are made, and B and T cells whose antibodies or TCR show a higher affinity for their respective targets are selected. If low affinity clones from the same lineage have neutralization function, the potency usually increases in clones with more mutation to acquire higher affinity.

Thus, in a further embodiment, the method comprises clustering amino acid sequences that have a sequence homology of 70%, 80%, or preferably 90% or more across a whole or part of said sequences. In another embodiment, the method comprises clustering amino acid sequences from a single clonal family cluster that have a sequence homology of 70%, 80%, or preferably 90% or more across a whole or part of said sequences. In a further embodiment, the method comprising clustering paired heavy and light chain sequences that have a sequence homology of 70%, 80%, or preferably 90% or more across a whole or part of the variable heavy or variable light chain region.

In some embodiments, the method comprises clustering related amino acid sequences based on at least one characteristic of the sequences, followed by clustering based on sequence homology, as described above.

As a result, clusters contain cells derived from the same precursor B or T cell. The greater the number of cells or sequences in a single cluster is indicative of a greater B or T cell clonal expansion.

### Lead Selection

The method of the invention comprises selecting a heavy or light chain lead sequence for antibody expression. In some embodiments, the method comprises selecting a heavy and light chain lead sequence for antibody expression.

The method of the invention is able to identify a lead antibody sequence from only a single sampling and sequencing step.

Lead sequences are selected from a cluster that contains more than one, preferably more than 2, more than 3, more than 4 or more than 5 paired antibody chain sequences, since this corresponds to B or T cells that underwent the greatest clonal expansion in response to the antigen. Said cluster must comprise at least one heavy or light chain sequence which is not from an intact cell. The method may comprise selecting a heavy or light chain lead sequence that is not from an intact cell.

Clusters may be analysed to determine the number of mutations in a heavy or light chain sequence and compared to their corresponding germline reference, i.e. the original precursor B or T cell sequence before it underwent clonal expansion. Sequences with the greatest number of mutations may be selected as a lead.

Thus, in some embodiments, the selected lead sequence comprises 1, 2, 3, 4, 5 or more mutations compared to the corresponding precursor B or T cell sequence.

In the case where a heavy and light chain sequence is being selected, said sequence is selected from the same cell i.e. the same intact cell or same reconstructed cell.

In some embodiments, the heavy or light chain lead sequence is selected from a heavy or light chain sequence that is not from an intact cell. In other embodiments, the heavy or light chain lead sequence is selected from a heavy or light chain sequence that is from an intact cell.

Once leads are selected, the sequences can be used to express an antibody. Further testing can then be carried out to determine whether the lead is suitable for further drug discovery testing.

The invention therefore also provides an antibody obtained by any of the methods disclosed herein.

The invention relates to a method as described herein, where the selected heavy and light chains are then expressed together in a cell to generate an antibody, or TCR, which may be optionally formulated with a pharmaceutically acceptable excipient or carrier, such as water, to form a pharmaceutical composition.

The invention also relates to a method as described herein, where one or both of the selected heavy and light chains are further modified, such as by truncation or conjugation, before being expressed together in a cell to generate an antibody, or TCR, which may be optionally formulated with a pharmaceutically acceptable excipient or carrier, such as water, to form a pharmaceutical composition.

The invention also relates to a method as described herein, where a heavy chain may be expressed alone in a cell to generate an antibody chain which may be optionally formulated with a pharmaceutically acceptable excipient or carrier, such as water, to form a pharmaceutical composition.

The invention also relates to a method of producing an antibody, the method comprising i) identifying a lead antibody sequence according to a method as described herein, ii) introducing the lead sequence into a host cell, iii) incubating the host cell to permit expression of the antibody, iv) recovering the antibody, and, optionally, v) purifying the antibody.

The invention also relates to a method of producing a nucleic acid encoding an antibody, the method comprising i) identifying a lead antibody sequence according to a method as described herein, ii) making a nucleic acid encoding the antibody, e.g., within a cell, such as within a production cell. Optionally, the nucleic acid is formulated as a pharmaceutical for delivery to a human or animal, or providing within a cell which is suitable for delivery to a human or animal body.

The invention also relates to a method of producing an T cell receptor, the method comprising i) identifying a lead TCR sequence according to a method as described herein, ii) introducing the lead sequence into a host cell, iii) incubating the host cell to permit expression of the TCR, iv) recovering the TCR, and, optionally, v) purifying the TCR.

The invention also relates to a method of producing a nucleic acid encoding T cell receptor, the method comprising i) identifying a lead TCR sequence according to a method as described herein, ii) making a nucleic acid encoding the TCR, e.g., within a cell, such as within a production cell. Optionally, the nucleic acid is formulated as a pharmaceutical for delivery to a human or animal, or providing within a cell which is suitable for delivery to a human or animal body.

### Hashtagging

The method of the invention may optionally further comprise providing the single sample of B or T cells that are labelled with oligonucleotide-tagged (oligo-tagged) antibodies or fragments thereof (disclosed herein as ‘hashtagging’). Said antibodies or fragments thereof may target ubiquitously expressed surface proteins of the B or T cells. Any suitable oligo-tagging technique in the art may be used in the methods disclosed herein.

Hashtagging the cell sample enables cells from different hosts, such as from different mice, to be pooled into the single sample for analysis. The method of the invention can then be carried out in a single step on a larger number of cells from different hosts, and the resulting cells in the clusters or lead sequences tracked back to the original host.

The term ‘binding’, ‘tagging’ and ‘associating’ will be used inter-changeably herein.

Therefore, the method disclosed herein further comprises tagging cells in the single sample with an oligo-tagged antibody, for example wherein the single sample is incubated with oligo-tagged antibodies, wherein the antibodies target a cell surface receptor on the B or T cells.

In some embodiments, the sample of B or T cells comprises cells from one or more different hosts, wherein the cells from each host are tagged with a different oligo-tagged antibody, for example different fluorescently labelled antibodies.

In some embodiments, the B or T cells are tagged in the cell mixture from the extracted tissue derived from the immunised host(s), i.e. before the B or T cells are sorted and encapsulated.

The inventors have also found that tagging the cells in this way can be used as an indicator of cell size. Larger cells are capable of binding more oligo-tagged antibodies compared to smaller cells, because of their larger cell surface area. Thus, cells with a high level of oligo expression indicates a larger cell, for example an intact cell, compared to a comparatively lower level of oligo expression which indicates a smaller cell, for example a fragmented cell. The level of oligo expression may be determined experimentally, depending on the type of oligo used. For example, when using a fluorescent oligo, the level of fluorescence may be measured to provide a size indication of each cell.

The level of oligo expression may be determined for cells in the sample, before the sample is processed, so that the ‘standard’ level of expression by be measured and used as a control for an intact cell.

This cell size indicator can be utilised at the sequencing stage of the method disclosed herein, to more accurately identify intact (larger) cells comprising a heavy and light chain pair, and fragmented (smaller) cells comprising unpaired heavy or light chains.

Thus, in some embodiments, the methods described herein further comprises providing a sample of B or T cells that are labelled with an oligonucleotide-tagged (oligo-tagged) antibody or fragment thereof, wherein at the sequencing stage, the level of oligo association for each cell in the sample is measured, to identify paired antibody heavy and light chain sequences from intact cells and nucleic acid sequences encoding antibody chains that are not from intact cells. In one embodiment, the measurement is compared to the level of oligo association for each cell in the sample before the sample is processed, for example by FACS/MACS sorting and encapsulation.

In a further embodiment, at the sequencing stage, the level of oligo association for each cell in the sample is measured and the level of V(D)J expression for each cell in the sample is determined, wherein these measurements are combined to identify paired antibody heavy and light chain sequences from intact cells and nucleic acid sequences encoding antibody chains that are not from intact cells. In some embodiments, the level of oligo association for each the cells in the sample is determined before the V(D)J expression.

In some embodiments, the method comprises preparing two next-generation sequencing DNA libraries from the oligo-tagged cell sample, after sorting and encapsulation, optionally from the barcoded encapsulates. The level of oligo association is analysed in the first library, and the V(D)J expression is analysed in the second library.

The combination of oligo expression and V(D)J expression results in a more accurate determination of which cells in the sample are intact, and which are fragmented. In this way, hashtagging avoids ‘overcounting’ intact cells that are actually fragmented cells. Further, as lysed cells are commonly not considered in prior art sequencing methods, hashtagging the cells allows accurate identification of intact cells, and prevents nucleic acid in the sample being discarded in situations where it was not previously clear whether it was from an intact or fragmented cell.

The inventors have also found that when correlating both the V(D)J expression and oligo association, it is also possible to identify non-B cells or non-T cells in the sample. It is also possible to identify B or T cells that have low numbers of heavy and light chains.

Non-B cells or non-T cells will have a relatively low level of oligo association compared to the B or T cells in the sample, because the non-B or non-T cells may not comprise the complementary cell surface receptor for binding the oligo-tagged antibody or fragment thereof. These cells may have been missed in the cell sorting stage. Further, B or T cells with low numbers of heavy and light chains will have a relatively low V(D)J expression levels, and so it is also possible to identify these cells based on the relative V(D)J expression levels of cells in the sample.

For example, when assessing the V(D)J expression and oligo association of the cells in combination, the following scenarios and information may be determined:
High hashtags counts and high VDJ counts for 1 heavy and 1 light: single cell encapsulation of a highly expressing B cell. This is an ideal result.
High hashtags counts and no VDJ counts: single cell encapsulation of a non-B cell. No data generated, but important to count as an intact cell and contrast to the estimated initial cell count.
High hashtag counts and low VDJ counts for only one chain: single cell encapsulation of a non-B cell with some ambient/debris from a B cell: the ambient chain will be used to enlarge candidate selection.
Low hashtag counts from 1 hashtag and medium/high VDJ counts for 1 heavy and 1 light: single cell encapsulation of a partially fragmented highly expressing B cell. If several of these are clustered, this is probably an original 1 single cell that fragmented into a handful of fragments.
Low hashtag counts from 1 hashtag and low/medium VDJ counts for 1 heavy and 1 light: single cell encapsulation of a partially fragmented low expressing B cell. If several of these are clustered, this is probably an original 1 single cell that expressed low levels of heavy/light chain but still relevant.
High hashtag counts from one hashtag, low counts from a second hashtag, and 1 heavy+1 light clear pair, with a third chain from the second hashtag: possible to identify that there has been an encapsulation of an intact cell from one hashtag, but some contaminant in the encapsulation from a fragment/debris of a second cell fragment from a different host, such as a mouse. Pairing of the correct heavy/light pair from the first hashtag is possible, whilst leaving the rest of material for ambient fraction.

Thus, the method disclosed herein further comprises providing a sample of B or T cells that are labelled with an oligonucleotide-tagged (oligo-tagged) antibody or fragment thereof, wherein after the sequencing step, the level of oligo association and V(D)J expression for each cell in the sample is measured, optionally wherein the levels of oligo association and V(D)J expression of each cell in the sample is assessed to determine the relative levels (i.e. high and low) of oligo association and V(D)J expression (e.g., cells in the sample may be compared with each other and/or with an external control), to identify paired antibody heavy and light chain sequences from intact cells and nucleic acid sequences encoding antibody chains that are not from intact cells.

Thus, the invention provides a method for identifying a lead antibody sequence, the method comprising:
i. providing a single sample of B cells derived from a spleen and/or bone marrow tissue, wherein the sample comprises intact and fragmented B cells;
ii. performing a single sequencing step to sequence nucleic acid from the single sample, to identify paired antibody heavy and light chain sequences from intact cells and nucleic acid sequences encoding antibody chains that are not from intact cells, and
   selecting a heavy or light chain lead sequence for antibody expression, wherein the heavy or light chain lead sequence forms part of a cluster of homologous sequences, and the cluster comprises at least one heavy or light chain sequence which is not from an intact cell,
   wherein in step i) the cells in the single sample are bound to an oligo-tagged antibody or fragment thereof,
   optionally
   wherein the single cell sample is from spleen and/or bone marrow tissue from one or more hosts, wherein the tissue from each host is associated with a different oligo-tagged antibody, and/or
   wherein step ii) further comprises determining the level of oligo association for each cell in the single sample is measured, to identify paired antibody heavy and light chain sequences from intact cells and nucleic acid sequences encoding antibody chains that are not from intact cells, and/or
   wherein step ii) further comprises determining the level of oligo association and V(D)J expression for each cell in the single sample, optionally wherein the levels of oligo association and V(D)J expression of each cell in the single sample is assessed to determine the relative levels (i.e. high and low) of oligo association and V(D)J expression, to identify paired antibody heavy and light chain sequences from intact cells and nucleic acid sequences encoding antibody chains that are not from intact cells.

The invention also provides a method for identifying a lead T cell receptor (TCR) sequence, the method comprising:
i. providing a single sample of T cells derived from a thymus and/or bone marrow tissue, wherein the sample comprises intact and fragmented T cells;
ii. performing a single sequencing step to sequence nucleic acid from the single sample to identify paired TCR heavy and light chain sequences from intact cells and nucleic acid sequences encoding TCR chains that are not from intact cells, and
   selecting a heavy or light chain lead sequence for TCR expression, wherein the heavy or light chain lead sequence forms part of a cluster of homologous sequences, and the cluster comprises at least one heavy or light chain sequence which is not from an intact cell
   wherein in step i) the cells in the single sample are bound to an oligo-tagged antibody or fragment thereof,
   optionally
   wherein the single cell sample is from spleen and/or bone marrow tissue from one or more hosts, wherein the tissue from each host is associated with a different oligo-tagged antibody, and/or
   wherein step ii) further comprises determining the level of oligo association for each cell in the single sample to identify paired antibody heavy and light chain sequences from intact cells and nucleic acid sequences encoding antibody chains that are not from intact cells, and/or
   wherein step ii) further comprises determining the level of oligo association and V(D)J expression for each cell in the single sample, optionally wherein the levels of oligo association and V(D)J expression of each cell in the single sample is assessed to determine the relative levels (i.e. high and low) of oligo association and V(D)J expression, to identify paired antibody heavy and light chain sequences from intact cells and nucleic acid sequences encoding antibody chains that are not from intact cells.

### EXAMPLES

The following Examples describe exemplary protocols for carrying out steps of the method of the invention.

### Example 1: Sampling, Sequencing and Pairing B Cell Sample, Comprising Intact and Fragmented Cells

The spleen and bone marrow of each immunised mouse (immunised with a target of interest) of a cohort are extracted and a single-cell suspension is generated. These are stained with specific antibodies to B-cell markers together with antigen-specific probes and hashtag antibodies. The hashtag antibodies (e.g. BioLegend TotalSeq-C) bind to a ubiquitously expressed murine cell surface protein. Each of the antibodies contains a DNA hashtag which can be sequenced through Next-generation Sequencing methods. The cell material is then sorted through Flow Sorting or Magnetic sorting to select B cells and/or plasmablasts (plasma cells). The resulting sorted cells are counted, spun down for pelleting and processed through the microfluidics encapsulation procedure of the Genomics Chromium Single Cell Immune Profiling (10× Genomics), using the 5′VDJ kit (10× Genomics) and the manufacturer's protocol. After performing quality control (QC) on the resulting encapsulation, an estimate of the total amount of material is recorded and compared to the number of counted cells before encapsulation. In two parallel steps, two library preps are performed: (a) the 5′VDJ library prep kit is applied to the material resulting from encapsulation, using B-cell constant region primers for the mouse genome as provided in the 10× Genomics kit and spiking in the equimolar amount of dog constant lambda primers, designed equivalently to the inner/outer primers in the 10× Genomics kit; (b) the hashtag library is generated by size-selection from the post-encapsulation material, separating it from the DNA material in (a).

The 5′VDJ and hashtag libraries are sequenced as parallel samples in an Illumina NGS sequencer as 2×150 bp cycles, either on a MiSeq or NextSeq 550 or NovaSeq 6000. The 5′VDJ library should achieve a minimum of 5000 read pairs per cell, as recommended in the 10× Genomics protocol. The hashtag library should achieve a minimum of 1000 read pairs per cell, as recommended in the BioLegend TotalSeq-C protocol. The resulting sequencing data is demultiplexed and each library, both VDJ and hashtag, will contain a Read1 and Read2.fastq.gz pair of files.

The resulting set of FASTQ files is processed via the Cell Ranger software (10× Genomics): for the VDJ library, it is processed via ‘cellranger vdj’, given the list of heavy and light V+D+J+C reference sequences in the immunised mouse, such as the particular version of the Ky9 platform mice (described, for example in WO2018/189520) and with the estimated number of cells from the counting step as a parameter and the list of inner primers as a parameter. The results of the ‘cellranger vdj’ step are QCed and compared with the estimated number of cells in the cell counting step. The reconstructed chains from the ‘cellranger vdj’ step (all_contig.fasta) are blasted against the set of heavy and light V+D+J reference sequences formatted for running NCBI igblastn (National Centre for Biotechnology Information), with a total number of 10 results per query sequence. The results of the igblastn step are sorted by V_IDENTITY and filtered for the V_CALL sequence corresponding to the heavy+light V repertoire in the version of the Ky9 mouse platform, with only the highest V_IDENTITY hit chosen as the final result (set igbl). For the hashtag library, it is processed via the ‘cellranger count’ command in ‘feature-only’ mode, giving as input (a) the pair of .fastq.gz files for the hashtag library, (b) the list of hashtags in a feature_ref file, and (c) the transcriptome reference to the mouse genome (GRCm38). The resulting filtered barcodes list will contain the barcodes considered to be intact cells by this ‘cellranger count’ feature-only step.

The results of ‘cellranger vdj’ for the VDJ library and the ‘cellranger count’ feature-only for the hashtag library are fed as input to the 10× Genomics enclone software with both ‘DEFAULT’ (stringent) parameters (set DEF.ALL.isoc.encl) and ‘NCELL’ (lenient) parameters (set NCL.ALL.isoc.encl). The enclone software in DEF.ALL.isoc.encl will filter out any barcodes not present in the hashtag filtered barcodes list, ensuring that cells that are deemed fragmented from the hashtag library counts are taken into account by the enclone computation. Given the annotated table of enclone results, the following multi-step procedure is performed to classify the heavy+light sequence pairs for each cell barcode that passed enclone filters:

1) Take all cell barcodes from the enclone output where the heavy chain is in the chain1 output and the light chain is in the chain2 output, i.e. 1-2, where the heavy is in 1 and the light is in 2.

2) For the cell barcodes not present above, take the cell barcodes from 1-3.

3) For the cell barcodes not present above, take the cell barcodes from 1-4.

4) For the cell barcodes not present above, take the cell barcodes from 2-3.

5) For the cell barcodes not present above, take the cell barcodes from 2-4.

6) For the cell barcodes not present above, take the cell barcodes from 3-4.

From the sequence output of enclone NCELL (lenient) (set NCL.ALL.isoc.encl), the nucleotide sequences including the leader sequence but excluding the 5′ UTR (vj_seq1/vj_seq2), are translated into aminoacidic sequences using ‘seqkit translate--clean’. These are blasted against a formatted NCBI igblast-aa aminoacidic version of the references in the Ky9 platform (set igba). The results are combined in a strict inner join with the set igbl and the set igba for form the sets DEF.ALL.isoc.encl.enib and NCL.ALL.isoc.encl.enib.

From the NCL.ALL.isoc.encl.enib set, subtract the entries already present in DEF.ALL.isoc.encl.enib and list as a set of either heavy (set seq1) or light (set seq2) aminoacidic FASTA sequences file. In parallel, concatenate the aminoacidic heavy and light chains of set DEF.ALL.isoc.encl.enib in a fasta file (set scel). A CD-HIT-2D query is then performed where the input is the set scel and the input2 is the set seq1 or the set seq2, with a maximum number of outputs of 5 and an identity threshold of 0.9. The highest scoring result of CD-HIT-2D clustering for each sequence in set seq1 or set seq2 is taken, and assigned to the hit in set scel. For each assignment, the corresponding chain is copied from the hit in set scel as partner to the chain in seq1 (light chain of the best match in scel to seq1 chain) or seq2 (heavy chain of the best match in scel to seq2 chain), and these resulting paired sets are labelled as NCL.ALL.isoc.encl.ambi.seq1 and NCL.ALL.isoc.encl.ambi.seq2, tagging each record as amh (ambient heavy) or aml (ambient light).

The results of DEF.ALL.isoc.encl.enib and NCL.ALL.isoc.encl.ambi.seq1 and NCL.ALL.isoc.encl.ambi.seq2 are combined as the final set of paired entries

### Example 2: Clustering the Paired B Cells

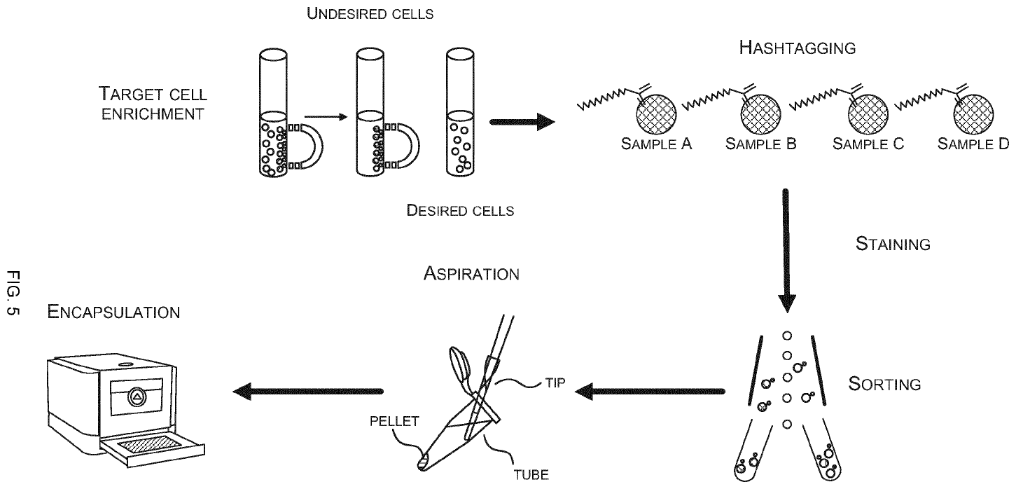

The sequences were analysed using custom tools based on the pRESTO/Change-O (Yale University)/Igblast (NCBI) software. The software predicts germline sequence and the hypermutation of the analysed IG sequence. The variable immunoglobulin region comprises a VDJ region of an immunoglobulin nucleotide sequence for heavy genes and a VJ region of an immunoglobulin nucleotide sequence for Igk and IgA. A clonal family is generally defined by the use of related immunoglobulin heavy chain and/or light chain V(D)J sequences by 2 or more samples. Related immunoglobulin heavy chain V(D)J sequences can be identified by their shared usage of V(D)J gene segments encoded in the genome. An example of the analysis of antibody sequences of sorted Ag-specific single B-cells is shown in WO2015/040401, FIG. 5. Here, the antibody sequences expressed by individual B cells were arranged by heavy-chain V-gene family usage and clustered to generate the displayed phylogenetic trees.

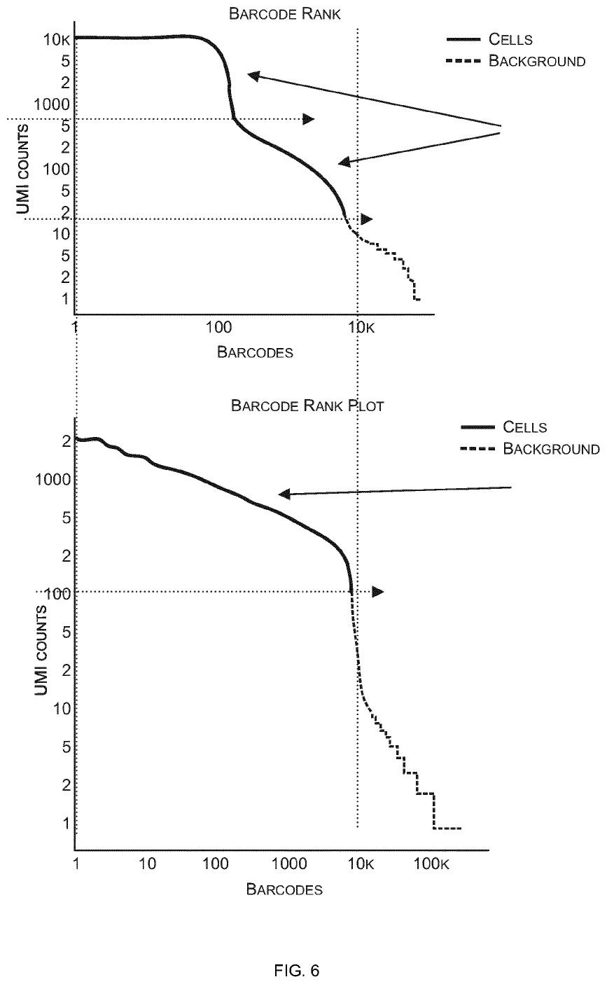

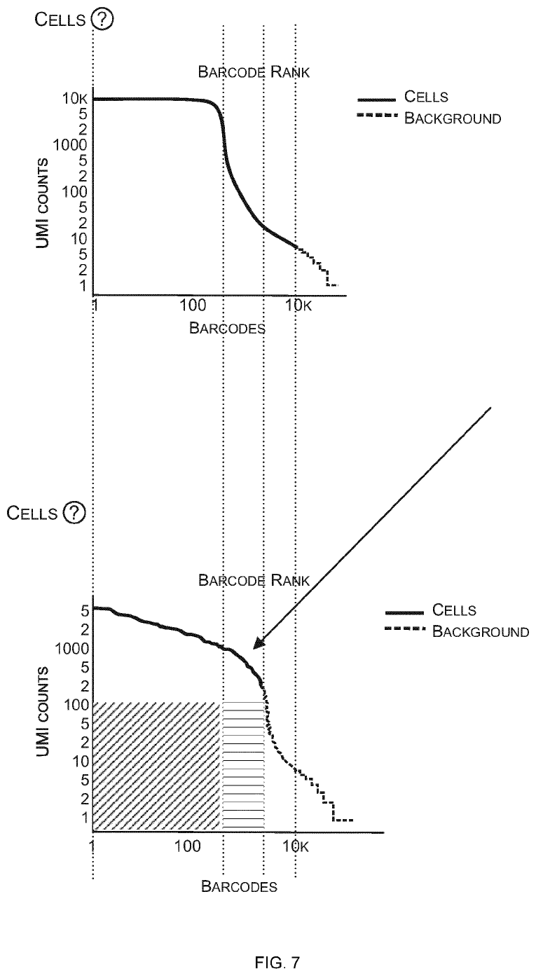

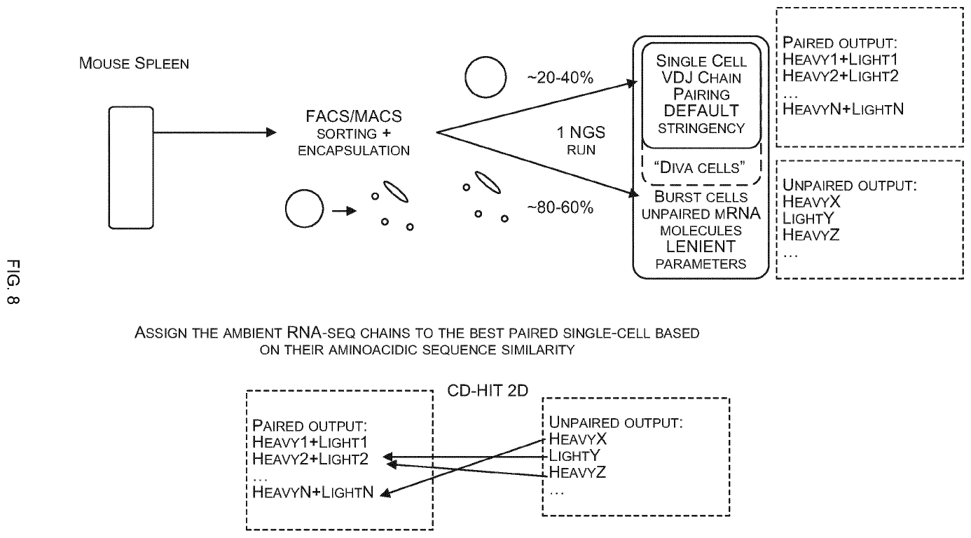

Within a clonal family, there are generally subfamilies that vary based on shared mutations within their V(D)J segments, that can arise during B-cell gene recombination and somatic hypermutation. Clones with different V(D)J segment usage usually exhibit different binding characteristics. Also, clones with the same V(D)J segment usage but different mutations exhibit different binding characteristics. B cells undergo somatic hypermutation, where random changes in the nucleotide sequences of the antibody genes are made, and B cells whose antibodies have a higher affinity B cells are selected (this is shown, for example, in an example clustered family in WO2015/040401, FIG. 6 which showed the affinity maturation via hypermutation for both apparent affinity and neutralization potency). If low affinity clones from the same lineage have neutralization function, the potency usually increases in clones with more mutation to acquire higher affinity.

### Example 3: Selecting Heavy and/or Light Chain Lead Sequences for Antibody Expression

The data for each sequence pair, cluster and phylogenies is loaded on a database and visualized on a webpage). The node graph of each clusters is coloured so that each node (cell) has a shade in a gradient of VH amino acid mutations, and the nodes (cells) with highest number of mutations to their corresponding germline reference are selected for synthesis.

The ambient mRNA molecules (amh and aml nodes or cells), which are tagged with respect to the rest of nodes (scel nodes or post-encapsulation single-cells) are considered as part of the selection process. If an ambient heavy or ambient light chain contains more mutations than other scel member chains of a phylogeny, the heavy+light pair stemming from the ambient node is selected for synthesis. This can increase the set of nodes (cells) in a given immunization cohort by up to 400% of the total number of scel nodes (post-encapsulation single-cells), depending on the amount of cells that have been determined as intact single-cells in the DEF.ALL.isoc.encl.enib set versus the number of chains from the excluded set between the DEF.ALL.isoc.encl.enib set and the NCL.ALL.isoc.encl.enib set.

1. A method for identifying a lead antibody sequence, the method comprising:
i. providing a single sample of B cells derived from a spleen and/or bone marrow tissue, wherein the sample comprises intact and fragmented B cells;
ii. performing a single sequencing step to sequence nucleic acid from the single sample, to identify paired antibody heavy and light chain sequences from intact cells and nucleic acid sequences encoding antibody chains that are not from intact cells; and
selecting a heavy or light chain lead sequence for antibody expression, wherein the heavy or light chain lead sequence forms part of a cluster of homologous sequences, and the cluster comprises at least one heavy or light chain sequence which is not from an intact cell.

2. A method for identifying a lead T cell receptor (TCR) sequence, the method comprising:
i. providing a single sample of T cells derived from a thymus and/or bone marrow tissue, wherein the sample comprises intact and fragmented T cells;
ii. performing a single sequencing step to sequence nucleic acid from the single sample, to identify paired TCR heavy and light chain sequences from intact cells and nucleic acid sequences encoding TCR chains that are not from intact cells, and
selecting a heavy or light chain lead sequence for TCR expression, wherein the heavy or light chain lead sequence forms part of a cluster of homologous sequences, and the cluster comprises at least one heavy or light chain sequence which is not from an intact cell.

3. The method of claims 1-2, wherein for the B cells, the spleen and/or bone marrow tissue is derived from a rodent that has been immunised with a target antigen and for the T cells, the thymus and/or bone marrow tissue is derived from a rodent that has been immunised with a target antigen.

4. The method of any preceding claim, wherein in step i), the B or T cells are sorted, optionally counted, and spun down for pelleting, and optionally wherein the cells are sorted via FACS or MACS.

5. The method of any preceding claim, wherein in step i), the intact and fragmented B or T cells of the single sample are encapsulated into emulsion particles, optionally into microfluidic drops, and wherein the sample comprises a mixture of encapsulated intact cells and encapsulated nucleic acid from the fragmented B or T cells, optionally wherein the encapsulated nucleic acid from the fragmented B cells encodes an antibody heavy or light chain and the encapsulated nucleic acid from the fragmented T cells encodes a TCR heavy or light chain.

6. The method of any preceding claim, wherein the nucleic acid in the sample is RNA.

7. The method of any preceding claim, wherein in step ii), the nucleic acid is sequenced via a next-generation sequencing instrument.

8. The method of any preceding claim, wherein after step ii) an antibody or TCR chain that is not from an intact cell is partnered with a heavy or light chain from a paired heavy and light chain from an intact cell, optionally further comprising comparing the amino acid sequences of the antibody or TCR chains that are not from intact cells with the paired antibody or TCR heavy and light chain sequences from intact cells, and selecting a heavy or light chain from a paired sequence to partner with a nucleic acid sequence encoding an unpaired antibody or TCR chain that is not from an intact cell, wherein the corresponding heavy or light chain from said paired sequence is at least 90% homologous to the amino acid sequence of the unpaired antibody or TCR chain.

9. The method of any preceding claim, wherein sequences that are derived from the same precursor B or T cell are clustered together in a single cluster, and/or wherein sequences with an amino acid sequence homology of 90% or more across the variable heavy or variable light domains are clustered.

10. The method of any preceding claim, wherein the cluster comprises at least one heavy and one light chain that are not from intact cells, optionally wherein the cluster further comprises at least one heavy and light chain from intact cells, or wherein the cluster further comprises at least one heavy and light chain from intact cells.

11. The method of any preceding claim, wherein the heavy or light chain lead sequence is selected from a heavy or light chain sequence that is not from an intact cell, or wherein the heavy or light chain lead sequence is selected from a heavy or light chain sequence that is from an intact cell.

12. The method of any preceding claim, further comprising expressing the heavy and light chains lead sequences together in a cell to generate an antibody or TCR, optionally further formulating with a pharmaceutically acceptable excipient or carrier to from a pharmaceutical composition.

13. The method of any preceding claim, wherein in step i) the cells in the sample are bound to an oligo-tagged antibody or fragment thereof, optionally, wherein the cell sample is from tissue from one or more hosts, wherein the tissue from each host is associated with a different oligo-tagged antibody.

14. The method of claim 13, wherein step ii) further comprises determining the level of oligo associated with each cell in the sample, to identify paired antibody heavy and light chain sequences from intact cells and nucleic acid sequences encoding antibody chains that are not from intact cells, and/or wherein step ii) further comprises determining the level of oligo association and V(D)J expression for each cell in the sample, optionally wherein the levels of oligo association and V(D)J expression of each cell in the sample is assessed to determine the relative levels of oligo association and V(D)J expression, to identify paired antibody heavy and light chain sequences from intact cells and nucleic acid sequences encoding antibody chains that are not from intact cells.

15. The method of any preceding claim, wherein the selecting comprises selecting a heavy or light chain lead sequence that is not from an intact cell.
